(19) United States
(12) Patent Application Publication
(10) Pub. No.: US 2020/0129412 A1
(43) Pub. Date: Apr. 30, 2020

(54) SILICONE PAD HAVING ACTIVE INGREDIENTS FOR SKIN CARE AND AGAINST SKIN AGING

(71) Applicant: Apricot GmbH, Baldham (DE)

(72) Inventor: Verena JÄGER, Forstinning (DE)

(73) Assignee: Apricot GmbH, Baldham (DE)

(21) Appl. No.: 16/620,869

(22) PCT Filed: Jun. 8, 2018
(86) PCT No.: PCT/DE2018/100548

§ 371 Date: Dec. 9, 2019

(30) Foreign Application Priority Data
Jun. 8, 2017 (DE) 10 2017 209 702.7
Jul. 7, 2017 (DE) 10 2017 211 702.8
Jan. 23, 2018 (DE) 10 2018 101 500.3

(51) Int. Cl. A61K 8/898; A45D 44/22; A61Q 19/08; A61K 8/02; A61K 8/73; A61K 8/65

(52) U.S. Cl. CPC A61K 8/898; A45D 44/22; A61K 8/65; A61K 8/0208; A61K 8/735; A61Q 19/08

(57) ABSTRACT

Summary

The present invention relates to a silicone pad for adhering to the skin, comprising at least one active ingredient for cosmetic skin care or against skin aging. In addition, the present invention relates to a cosmetic method for smoothing the skin, for the induction of scar remission, and it relates to the addition of active ingredients in the course in smoking cessation by means of applying a silicone pad to the skin of a person.

The present invention relates to a silicone pad comprising at least one active ingredient for the cosmetic skin care or for preventing skin aging as well as for releasing of medicinal or other active ingredients such as nicotine during smoking cessation. In addition, the present invention also relates to a silicone pad, comprising an adhesive layer, which consists essentially of silicone and comprises the active ingredient, and further comprising a silicone body.

Furthermore, the present invention also relates to a method for manufacturing a silicone pad comprising the step of applying an active ingredient to a silicone composition in order to produce a silicone pad. The present invention also relates to a cosmetic method comprising applying a silicone pad to the skin.

### BACKGROUND

Current silicone pads are based on the finding that wrinkles may be smoothed mechanically by longer duration of application, for example, overnight. The skin structure remains in the smoothed position for several hours and can thus regenerate. Such pads or adhesive bandages for adhering to the human skin in order to address wrinkling are well known. Mostly these are used on the décolleté.

The German utility model DE 20 2014 001 876 U1 discloses a décolleté-silicone pad for the reduction of wrinkles, which consists of 100% medical silicone (with dimensions of about 20×20×2.5 cm) and which has a self-adhering bearing surface. The property of self-adhering of the self-adhering bearing surface is obtained by special processing steps (hardness of the silicone and polished side as bearing surface). Therefore, the décolleté-silicone pad may be used for several months and does not lose its properties.

The Canadian Patent Application CA 2,575,534 discloses an anti-wrinkle device for use between the female breasts, at the décolleté as well as on the upper chest, which comprises a semi-rigid/semi-flexible membrane, a soft layer and a further adhesive layer.

The German utility model DE 202015106000 U1 discloses a décolleté-Pad for adhering to the skin having a support layer and an adhesive layer. Preferably, the décolleté-Pad exhibits mirror symmetry along an axis of symmetry and is teardrop-shaped.

Disadvantageously, above devices merely effect a purely mechanical smoothing of the skin, such that its effect may wear off immediately after termination of the application and removal of the device from the skin. Additionally, such devices serve exclusively as anti-wrinkling or as anti-aging products, but are not utilized as skin care products or for reducing liver spots and pigmentation marks, cellulite, acne, atopic dermatitis or other skin disorders. A further disadvantage of these devices lies in their applicability to a specific area of the skin only, particularly to the décolleté and chest area, but not to other areas of the skin, for which a care effect as well as control and prevention of skin aging is desired also.

Moreover, the translation of European patent DE 695 16 553 T2 discloses a hot-melt silicone pressure sensitive adhesive comprising siloxylated polyether waxes as an additive. The melt-silicone PSA compositions disclosed herein exhibit hydrophilic properties while maintaining PSA properties in terms of shear, adhesion and peelability.

The german disclosure DE 101 14 382 A1 relates to a silicone-based moisture-absorbing matrix, in particular for wound care and/or pharmaceutical skin treatment, wherein the pressure-sensitive adhesive matrix consists of silicone, a gelling agent and optionally a silicone resin.

### INVENTION, OBJECT OF THE INVENTION, SOLUTION, ADVANTAGES

Based on the considerations of the prior art as mentioned above, the object of the present invention therefore is to provide a silicone pad which overcomes the disadvantages of the prior art as described above. The object underlying the invention is achieved by the silicone pads as defined in the claims, which also is apparent from the accompanying embodiments. In particular, the object of the present invention is the provision of a silicone pad, which supplies, over an extended period of time, an active ingredient incorporated into the silicone pad to the state.

Surprisingly, the inventors of the present invention have discovered that the active ingredient is able to diffuse from the silicone pad to the skin, is taken up by the latter and may develop a biological activity therein. This form of skin treatment is used particularly advantageously both in anti-aging treatment or for regular care as well as for treating liver spots and pigmentation marks, cellulite and other skin disorders such as wound healing or atopic dermatitis. Additionally, other active ingredients may be incorporated, such as pharmaceutical agents or nicotine for the cessation of smoking. Thus, the skin is mechanically flattened not only due to the weight of the silicone pads, but they also release active ingredients whose contribution to skin regeneration or to the treatment of the disease is particularly beneficial.

Surprisingly, the active ingredients may continue to exert effects within the skin even after cessation of treatment. Furthermore, by combining the silicone pad with the active ingredient, the contact and absorption capacity of the skin thus flattened with the silicone pad may be increased and thus ideal conditions are obtained for absorption of the active ingredient into the skin. In between the silicone pad and the respective skin layer a microclimate is generated, which, among other things, helps the skin to open up pores, which stimulates metabolic processes and which facilitates absorption of active ingredients.

The skin, having been flattened ideally for hours, is also in an optimal receptive state due to the silicone pad, since the dermal layer relived and therefore blood supply is significantly improved. Thus, robust and healthy skin cells may be generated, exhibiting good absorption capacity for the active ingredients. In this ideally flattened position, the skin is supplied with important active ingredients in the long term and may thus better regenerate and a smoothing of wrinkles may be achieved. Also, with respect to liver spots and pigmentation marks, the long-term application of the active ingredients by means of incorporating them into a silicone pad exerts an extremely positive effect on the skin, since active ingredients penetrate for a longer time and more intensely, thus achieving an optimal effect.

The silicone pad according to the present disclosure may also be utilized for treatment of scars. Here, the silicone pad provided with at least one active ingredient may be applied to scars, thereby stimulating the skin to achieve, via a microclimate, self-healing and faster cell renewal.

The present invention may be applied to all areas of the skin due to the different shapes in the various embodiments, especially to the back of a hand, to the neck, the décolleté, the forehead, under the eyes and around the mouth. It also may be applied to the upper arms, the lower chin and the pubic region, where cesarean sections are generally located.

In a first aspect, the present disclosure relates to a silicone pad (1) suitable for adhering to the skin. Said pad may contain at least one active ingredient for cosmetic skin care or for preventing skin aging. In addition, the silicone pad (1) may comprise a combination of several active ingredients.

For the purposes of the present disclosure, a silicone pad is a flat object largely consisting of medical grade silicone (medical silicone), which object is configured with dimensions in two spatial directions which are significantly larger than the dimension in a third spatial direction. The significantly larger dimensions in two spatial directions may lead to different shapes, in particular to a circle or different circular shapes with curves, to an ellipse, a polygon, a drop or to a hand shape.

The silicone pad is preferably an addition-crosslinked 2-component silicone, which is particularly suitable for making a silicone pad with good release characteristics regarding the active ingredient. This material comprises vinyl-terminated polydimethylsiloxanes (CAS No: 68083-19-2) and other cross-linking agents. The proportion of silicone is >95%. Preferably, the silicones are room-temperature-crosslinking, platinum-catalyzed silicone elastomers.

Preferably, the silicone is characterized by a high gas and water permeability. Thereby, the active ingredient may be transported optimally through the silicone pad. In an alternative implementation, the silicone pad is substantially permeable to gas, especially permeable to water vapor, but not easily penetrated by liquid water (waterproof).

Preferably, the silicone is an addition-crosslinking silicone that has cured to a soft, sticky silicone adhesive. To the additional advantageous properties of this silicone belong a very good biocompatibility, absence of by-products, good adhesion and good platinum crosslinkability.

An active ingredient is an endogenous or exogenous substance which is able to initiate or influence a change in a biological process, such as skin aging. An active ingredient may also be a pharmaceutical agent. As a rule, its absorption via the skin is advantageous for the user.

Due to the active ingredient capable of penetrating into the skin during treatment with the silicone pad, the skin is not only flattened and cared for mechanically due to the weight and the adhesion of the silicone pad (1), but also is treated by means of the active ingredient, wherein, due to the flattening, symbiotic effects may be observed with regard to pharmacokinetics.

It is particularly advantageous that the skin continues to undergo treatment by that fraction of the active ingredient already penetrated into the skin, even after removal of the silicone pad (1) from the skin, thereby providing additional skin care. Consequently, improved skin care as well as anti-aging effects may be achieved. The combination of silicone pad and active ingredient also allows for a maximal area of contact between the skin and the silicone pad and a favorably increased absorption of the active ingredient into the skin.

The components of the active ingredients may penetrate into the tissue via the skin and unfold therein their biological effect. The silicone pads according to the present disclosure exhibit no negative effects, in particular, wearing of the silicone pad does not create discomfort and there are no peel effects. The silicone pad feels soft to the touch, wrinkles are reduced visibly and skin compatibility is particularly good.

Without adhering to any particular theory, the inventors of the present invention believe that due to the high polarity of hyaluronic acid and other active ingredients and the low polarity of silicone, the hyaluronic acid or other respective active ingredients embedded in the adhesive gel will be delivered slowly to the epidermal layer of the skin, which rests on the dermal layer, such as to replenish hyaluronic acid already delivered to the dermal layer. In addition, other parts of the active ingredient will also migrate towards the basal stratum since there is no barrier layer. Comparative studies have shown that oil nebulized in water coalesce as oil droplets after some time. the

Without adhering to any particular theory, according to the inventors, particle release is it effected significantly by the heat and by the moisture of the skin, generating a microclimate beneath the silicone pad. Preferably, the silicone pad has a thickness of 0.5 to 2.0 mm, more preferably of 0.8 to 1.5 mm, in particular substantially 1.2 mm Such a thickness of the silicone pad provides for ideal wearing comfort and optimal imparts exposure to the active ingredient.

The concentration of the active ingredient in the silicone pad preferably is in the range of 0.5 to 3.0‰ (w/w), more preferably in the range of 1.0 to 2.0‰ (w/w is), and ideally is about 1.6‰ (w/w).

In an alternative implementation, the active ingredient may also be present in the silicone pad at a concentration in the range of 0.1 to 9.9%; preferably in the range of 0.5 to 7.0% and ideally of about 1.0% (w/w). In this case, oils such as coconut oil, olive oil or aloe vera in liquid form may also be used as active ingredient. Surprisingly, the silicone pad softens under these circumstances, providing the added benefit of good adhesion to the skin.

Advantageously, the silicone pad may also contain a silicone oil. If the silicone pad comprises a silicone oil, the pad is softer during application, providing for good adhesion to the skin. Silicone oils to be utilized within the meaning of this disclosure preferably are polymerized siloxanes having organic side chains. Therein, the side chains are characterized by periodic, alternating arrangement of silicon and oxygen atoms with the general empirical formula [R¹R²SiO]ₙ. The free outer electrons of the silicon are preferably attached to residues R, which mostly are organic residues but also may be halogens. The molecular weight of the silicone oils utilize preferably is in the range off 162 to 150,000 g/mol. The density of the silicone oils preferably is in the range of 0.76 to 1.07 g/cm³.

In an advantageous implementation, the silicone oils are characterized by a viscosity in the range of 0.6 to 1,000,000 mPs. As a result, the silicone pads possess optimal haptic properties.

The silicone oil preferably is polydimethylsiloxane in which the respective residues R¹ and R² are methyl groups.

In a preferred implementation, the silicone pad (1) may comprise an adhesive layer (2) and a silicone body (3). The adhesive layer (2) may consist essentially of silicone and may comprise the active ingredient.

Alternatively, the silicone pad (1) may consist of a silicone body (3) comprising the active ingredient. The silicone pad may be configured with a polished side. In this case, the silicone pad is characterized by a self-adhesion lasting over a longer time scale. The silicone pad or adhesive layer may include further auxiliary means allowing the silicone pad to be securely attached to the skin. For example, the silicone pad may comprise a cotton fabric on one side, which protects the silicone pad from external influences. Furthermore, the auxiliary means may be a slip-on cotton glove covering the back of the hand.

An adhesive layer is any layer of material that, after attaching the silicone pad to the skin, provides for its adherence to the skin for at least three hours or more without any additional application of force. In particular, an adhesive layer may consist essentially of silicone. Preferably, the adhesive layer is characterized by a silicone content of at least 95%.

Preferably, the silicone body is configured with a thickness in the range of 0.5 to 2.0 mm, more preferably in the range of 0.8 to 1.5 mm, in particular of substantially 1.2 mm. The adhesive layer preferably has a thickness in the range of 0.2 to 1.0 mm, particularly preferably in the range of 0.4 to 0.8 mm, in particular the range of 0.6 to 0.7 mm By providing such thickness of the silicone body and the adhesive layer, the amount (dosage) of the active ingredient is justifiable economically while at the same time the comfort of wearing the silicone pad is ideal.

Preferably, the adhesive layer of the silicone pad is softer than the silicone body. Moreover, compared to the silicone body, water permeability of the adhesive layer is higher. Under these circumstances, the silicone body of the silicone pad may be used as a mechanical support matrix while at the same time the active ingredient may be delivered reliably to the skin by the adhesive layer.

The concentration of the active substance in the adhesive layer preferably is in the range of about 0.6 to 5.0‰ (w/w), more preferably in the range of about 1.5 to 3.0‰ (w/w), and ideally is about 1.7‰ (w/w). Such concentration of the active ingredient in the adhesive layer provides for an ideal diffusion rate relative to material consumption of the active ingredient.

If the adhesive layer (2) of the silicone pad (1) comprises the active ingredient, maximum drug delivery to the underlying skin may be guaranteed. During application, the adhesive layer preferably may be in direct contact with the surface of the skin, thus enabling even faster, more intensive and longer absorption of the active ingredient by the skin. This allows a particularly effective treatment of the skin. Additionally, no active ingredients remain unused due to their retention within the silicone pad (1). The silicone pad in this embodiment is particularly advantageous in that the required stability and the necessary weight are provided for by the silicone body (3) in order to smoothen the skin mechanically. At the same time, the dosage of the active ingredient is particularly economical.

In a further implementation, with respect to its shape and size, the silicone pad (1) may be suitable for resting on a back of a hand (4).

The silicone pad (1) which is suitable for resting on the back of a hand preferably may exhibit a shape with fingers, wherein the silicone pad (1) may extend over the entire back of the hand (4) as wells as beyond the metacarpophalangeal joints (5) to the proximal interphalangeal joints (6). As a result, the skin area of the back of the hand (4) may be treated optimally. Furthermore, the silicone pad (1) may be shallower in the areas of the metacarpophalangeal joints (5) than in the remaining area of the back of the hand (4). This provides for more adaptability, flexibility and extensibility of the silicone pad (1) in these areas, such that the silicone pad (1) does not detach from the hand despite of its movement, but provides for optimum grip. This is advantageous, as the silicone pad (1), when compared to products already known, may not only be applied to the décolleté, the neck or the face, but also to other areas of the skin, such as the back of a hand. By way of employing in silicone pads with active ingredients for application to the neck, décolleté or face considerable advantages are achieved in the case of skin care and stimulation of regeneration and wrinkle reduction compared to previously known silicone pads with only manual skin tightening.

In a further preferred embodiment, the active ingredient of the silicone pad (1) may be selected from the group consisting of nutrients, in particular vitamins, lipids, and amino acids; polypeptides; antibiotics or their prodrugs; antioxidants; coenzymes; polysaccharides or hydroxycarboxylic acids.

In conjunction with the adhesive layer (2) of the silicone pad (1), these groups exhibit particularly advantageous effects. In particular, these active substances are able to diffuse exceptionally well from the silicone pad (1) towards the skin surface, to be absorbed by the skin and then to display their activity. For example, vitamins and antioxidants contribute to counterbalance free radicals and thus to anti-aging effects; amino acids, polypeptides and lipids bolster skin function and render it supple.

Furthermore, the active ingredient of the silicone pad (1) may be selected from the group consisting of vitamin A; vitamin C; vitamin E; vitamin B3; vitamin B5; Vitamin B9; vitamin B12; Coenzyme 10; arbutin; high and low molecular weight hyaluron; high and low molecular weight hyaluronic acid; collagen; b-resorcinol; 4-n-butylresorcinol; glycolic acid; salicylic acid; spirulina; chlorella; caviar extract; dimethylaminoethanol; shea butter; coconut oil; phytohormones, such as soybean phytohormones, hops, red clover or yam root (dioscoreaceae); phytoestrogens; isoflavones; lignans; amino acids, such as arginine, methionine, glutamine or creatine; oligopeptides; polypeptides; pentapeptides; glycerol; alpha-lipoic acid; or polyphenols, such as ginkgo, resveratrol from grapes or aloe vera.

Particularly preferably, the active ingredient of the silicone pad (1) is selected from the group consisting of hyaluron, collagen or aloe vera. Ideally, the active ingredient is hyaluron. Silicone pads with these active ingredients, especially hyaluronic acid, show excellent anti-wrinkling properties.

In a preferred implementation, the active ingredient of silicone pad (1) is selected from the group consisting of moringa powder, alpha lipoic acid, selenium, saffron extract, curcumin, turmeric, astaxanthin, blood orange, lingzhi mushroom, apple cider vinegar, lemon juice, ascorbic acid or reservatrol. Surprisingly, if the active ingredient is selected from this group of compounds, the silicone pad can be used to remove age spots.

In another preferred embodiment, the active ingredient of the silicone pad (1) is selected from the group consisting of caffeine; yam; jojoba, jojoba oil; wheat germ, wheat germ oil; Q10; green algae extract; ESP; menthol; retinol; aminophylline; forskolin; carnitine; aescin; panthenol; provitamin B5, B1, B2, B6, niacin, biotin; L-tyrosine; tocopheryl; mannitol; birch, birch water, birch oil or L-arginine. Surprisingly, if the active ingredient is selected from this group of compounds, the silicone pad may be used to remove cellulite.

In another preferred implementation, the active ingredient of the silicone pad (1) is selected from the group consisting of beta-carotene, omega-3, bentonite, tea tree oil, rose water, silica gel, swedish herbs, swedish bitters. Surprisingly, if the active ingredient is selected from this group of compounds, the silicone pad may be used to remove acne.

In a further preferred implementation, the active ingredient is present in the form of a cream or is an active substance that may be applied in the form of creams. Preferably, the active ingredient is selected from the group consisting of guar gum extract; currant; acerola; extracts of the African baobab tree; meadow seed oil; peptides; spinach extract; evening primrose oil; lipids such as cardiolipin; antioxidants such as 1-carnosine; rhodiola complex; lentil seed extract; star fruit extract; java tea extract or retinol. If the active ingredient is selected from this group of compounds, surprisingly, the silicone pad is particularly useful for cosmetic treatment of the skin.

In a preferred implementation, active ingredient of the silicone pad (1) is selected from the group consisting of carnea; linoleic acid; Omega 3; fenugreek; ginger; sango coral; colloidal silver; silica; spirulina; powder from green tea extract; carotene or rice protein. Surprisingly, if the active ingredient is selected from this group of compounds, the silicone pad may be used to remove atopic dermatitis.

In a preferred implementation, active ingredient of the silicone pad (1) is selected from the group consisting of aloe vera; apple cider vinegar; honey; cocoa butter; sandalwood; tea tree oil; kerria, kerria oil; licorice root; lugol's solution; lavender, lavender oil, rosehip seed, rosehip seedoil, arnica, anica Oil; sunflower; onion extract, allantoin, heparin; calendula; emu oil; zinc, vitamin C, beta-carotene; shea butter; coconut powder, coconut oil; yarrow; raspberry leaves, lavender; sandalwood in combination with rose water or St. John's wort. Surprisingly, if the active ingredient is selected from this group of compounds, the silicone pad may be used particularly well for treating wounds or scars.

In a further preferred implementation, the active ingredient of the silicone pad (1) is selected from the group consisting of argan oil; glutamine; L-arginine; L-carnitine; magnesium; turmeric; silica; avocado; rose water; grape seeds; ceramides; folic acid or soy. Surprisingly, if the active ingredient is selected from this group of compounds, the silicone pad may be used to remove wrinkles.

In another preferred implementation, the active ingredient of the silicone pad (1) is nicotine. In this case, the silicone pad may be used in the course of nicotine withdrawal. Compared to an absorption via the gastrointestinal tract, absorption via the skin confers less stress to the body and is also faster and more efficient.

In a further preferred embodiment, the active ingredient of the silicone pad is a pharmaceutical ingredient to be utilized for treating diseases or in the context of a hormonal therapy, for example using estrogen and/or progestogens for the inhibition of ovulation. Compared to an absorption via the gastrointestinal tract, absorption via the skin confers less stress to the body and is also faster and more efficient.

In addition to the above-mentioned active ingredients, the silicone pad or the adhesive layer of the silicone pad may also comprise other ingredients. For example, the silicone pad or the adhesive layer of the silicone pad may comprise one or more of the following other ingredients: neroli; neroli oil; rosemary; rhealba; copper sulfate, copper, zinc sulfate, zinc; glycerol; comfrey; witch hazels or peptides.

In a further aspect, the disclosure comprises a method for manufacturing a silicone pad (1) comprising the following step: applying an active ingredient to a silicone composition for producing a silicone pad (1).

This is advantageous, in that, after adhering the silicone pad (1) to the skin, the active ingredient is in direct contact with the skin surface and thus may be absorbed therefrom even faster, with higher intensity and for a longer time.

The disclosure further comprises a method for manufacturing a silicone pad (1) comprising two consecutive steps as follows: a) mixing an active ingredient with a silicone composition to produce an adhesive layer (2), and b) applying the adhesive layer to a silicone body (3) to produce a silicone pad (1).

The active ingredient may be processed as a fine powder or as a liquid or gel-like mass.

In a further aspect, the disclosure relates to a silicone pad (1) which is, with regard to its shape and size, suitable for resting on a back of a hand (4), the silicone pad being suitable for smoothing wrinkles on the back of the hand (4).

The silicone pad (1) which is suitable for resting on the back of a hand (4) may exhibit the shape of fingers, wherein the silicone pad (1) extends over the entire back of the hand (4) as well as beyond the metacarpophalangeal joints (5) up to the finger proximal interphalangeal joints (6). As a result, the skin area of the back of the hand (4) may be treated optimally.

In a further implementation, the silicone pad (1) may include hinge portions (7). A hinge portion is a subregion of the silicone pad which exhibits a reduced thickness of material and which thus is easier to fold compared to other subregions of the silicone pad.

This allows for improved adaptability, flexibility and extensibility of the silicone pad (1) in these areas, such that, despite the movement of the hand, the silicone pad (1) does not detach therefrom, but provides for optimum grip.

In another aspect, the disclosure further relates to a cosmetic method comprising applying to the skin a silicone pad according to the invention. By the cosmetic method, the skin is treated mechanically and tightened on the one hand, while on the other hand, the active ingredient of the silicone pad may penetrate into the skin tissue and cause a cosmetic effect therein.

As a result, prevention and reduction of wrinkles as wells of liver spots and pigmentation marks may be achieved. Additionally, the silicone pad may be applied in skin care, for wound and scar treatment, for skin diseases, cellulite or, in general, for the delivery of pharmaceutical agents. In different implementations, the silicone pad may be applied to different parts of the skin, especially to the back of a hand.

In addition, the disclosure also relates to a cosmetic method for inducing scar remission in a subject, comprising applying to the skin of the subject a silicone pad according to the present invention.

Furthermore, the disclosure also relates to a medical wound care procedure comprising applying to the skin a silicone pad according to this disclosure. By way of the medical procedure an open or closed wound in the skin is treated and cured.

Furthermore, the disclosure also relates to a medical treatment of nicotine dependence in a subject comprising applying to the skin a silicone pad according to this disclosure containing nicotine as an active ingredient. The medical treatment is used to treat the subject's nicotine dependence.

The silicone pad may contain other medical agents as active ingredients. Exemplary medicinal ingredients include antibiotics, proton pump inhibitors, antidepressants, analgesics, antihistamines, tranquilizers, antihypertensive agents, and other medicinal ingredients.

### SHORT DESCRIPTION OF THE FIGURES

Hereinafter, by way of example and not subject to any limitation, particular embodiments of the invention will be described with reference to the accompanying drawings.

The particular embodiments are merely illustrative of the general inventive concept, but do not limit the invention.

It is shown:

in FIG. 1, a lateral cross section of a silicone pad (1).

in FIG. 2, a plan view of a silicone pad (1) exhibiting the shape of fingers, suitable for resting on the back of a hand.

in FIG. 3, the result of drug absorption studies.

FIG. 1 shows a lateral cross-section of a silicone pad (1). The silicone pad (1) consists of a silicone body (3) and an adhesive layer (2) which is provided with active ingredients and which rests on the skin.

FIG. 2 shows a plan view of a finger-shaped silicone pad (1) exhibiting the shape of fingers, suitable for resting on a back of a hand (4). The silicone pad (1) covers the back of the hand (4) and extends beyond the metacarpophalangeal joints (5) to the proximal interphalangeal joints (6). The hinge region (7) are arranged directly above the metacarpophalangeal joints (5).

FIG. 3 shows the result of drug absorption studies. FIG. 3A depicts a silicone pad with an adhesive layer. The adhesive layer comprises 6 mg of powdered hyaluron (<20 T Dalton). The powder was stained with a green food coloring in order to demonstrate diffusion. For manufacturing a silicone pad, the adhesive layer was bonded to a silicone body. The silicone pad was applied daily to the skin of a subject for a period of 2 weeks.

It can be derived from this study, that the hyaluron does not accumulate in the upper layer, but was delivered to the skin's lower layers.

Additionally, one may derive from this study that the upper silicone layer functions as support layer. That is, it is characterized by a different strength and is essentially completely impermeable to water. On the other hand, the lower layer with the active ingredient, is softer and exhibits sufficient water permeability in order to deliver particles to the skin via skin contact (heat and moisture).

FIG. 3B shows the same silicone pad after 2 weeks of daily use. As may be seen clearly in FIG. 3B, the concentration of hyaluron in the silicone pad decreases markedly due to the diffusion into the skin, which is evidenced by a marked decrease of color of the silicone pad.

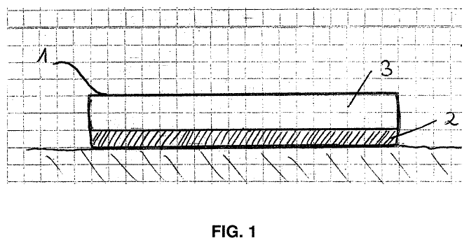

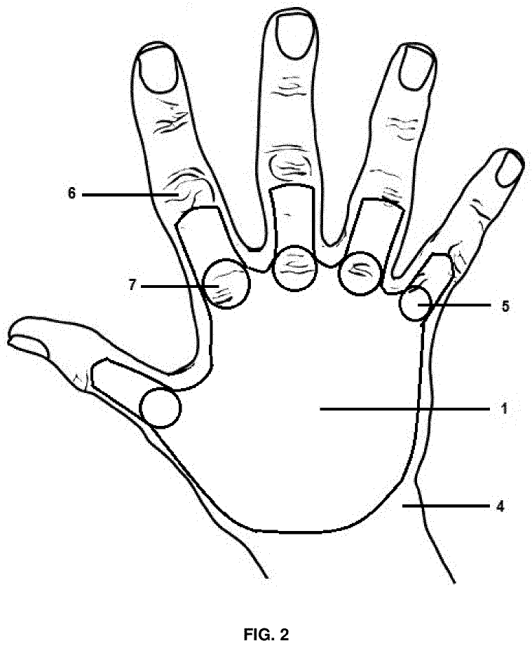

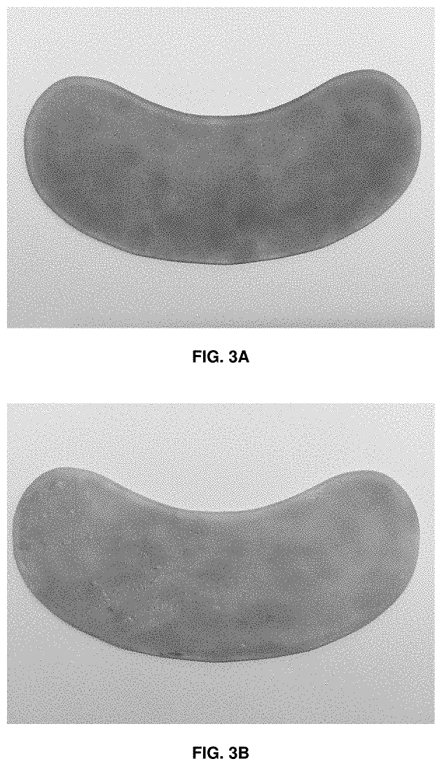

### EXAMPLES

Manufacture of Silicone Pads

In the following, by way of example, the manufacturing of silicone pads is described, which may be used as a décolleté pad or as an eye pad.

Information Concerning the Manufacturing Process

The material used contains vinyl-terminated polydimethylsiloxanes (CAS No: 68083-19-2) and other auxiliaries for crosslinking. The silicone content is >95%.

The 2-component, addition-crosslinking basic material is metered out evenly into the appropriate mold utilizing a computer-controlled multicomponent metering system. Subsequently, the mold is moved into an oven in which it is subjected to vulcanization by radiant heat for a period of about 5-7 min and at a temperature of about 55-65° C. In the next step, the 2-component, addition-crosslinking, sticky adhesive gel is applied uniformly to the hardened basic material as a thin layer using another computer-controlled multicomponent metering system. This mold also is moved back into an oven in which the adhesive gel also undergoes vulcanization by radiant heat. The vulcanization process runs over a period of approx. 8-10 min. at a temperature of about 70-85° C. The finished products are then removed from their molds, checked and packaged.

Substances (hyaluron, aloe vera . . . ) to be added, whether as liquids or powders, are mixed with a component of the adhesive gel utilizing a mixer run at a speed of up to 25,000 rpm in order to ensure homogeneous incorporation. In order to prevent sedimentation or segregation of the substance, the silicone component is stirred repeatedly according to a fixed schedule, depending on the substance stirred in, such that consistent quality may be ensured.

### Example 1: Décolleté Pad

Silicone pads were prepared from vinyl-terminated polydimethylsiloxanes (CAS No.: 68083-19-2) using conventional crosslinking agents as auxiliaries. According to the manufacturer's instructions, the silicone content is at least 95%. The production of these silicone pads has been described already elsewhere and corresponds, for example, to the silicone-based matrices disclosed in DE 101 14 382 A1. The silicone body of the silicone pads has the following values in (basic material): thickness 1.2 mm, weight 19.3 g. The adhesive layer has the following values: thickness 0.6 mm, weight 8.5 g. The weight fraction of added hyaluronic acid in the adhesive layer is 14 mg/8.5 g, thus is 1.6‰ (w/w).

The adhesive layer is prepared by stirring the powdered hyaluron (11-14 mg) into the silicone gel (8.5 g).

The silicone pad described above is suitable for use as a décolleté pad.

### Example 2: Eye Pad

Silicone pads were prepared from vinyl-terminated polydimethylsiloxanes (CAS No.: 68083-19-2) using conventional crosslinking agents as auxiliaries. According to the manufacturer's instructions, the silicone content is at least 95%. The production of these silicone pads has been described already elsewhere and corresponds, for example, to the silicone-based matrices disclosed in DE 101 14 382 A1. The silicone body of the silicone pads has the following values in (basic material): thickness 1.2 mm, weight 19 g. The adhesive layer has the following values: thickness 0.7 mm, weight 9 g. The weight fraction of added hyaluronic acid in the adhesive layer is 1.5 mg/0.9 g, thus is 1.7‰ (w/w).

The adhesive layer is prepared by stirring the powdered hyaluron (1.5 mg to 2.0 mg) into the silicone gel (0.9 g).

The silicone pad described above is suitable for use as an eye pad.

### Example 3: Efficacy Study

In an empirical efficacy study, silicone pads according to the invention and a blank were tested in a total of 15 subjects aged 29 to 61 years. The silicone pads listed in Table 1 were tested.

| Silikonpad No. | ingredients |
|---|---|
| 1 | — |
| 2 | hyaluron |
| 3 | collagen |
| 4 | aloe vera |

The silicone pads were brought into contact with skin for at least 1 hour/day on at least 14 consecutive days, ideally overnight. Contact surfaces were the back of a hand, the region below the eyes, the décolleté, neck and forehead. Thereafter, the subjects were asked for their subjective opinion, a) whether the silicone pads were tolerable and could be used without problems, b) whether a visible wrinkle reduction and/or other positive effects were observed, and c) which of the above silicone pads 1 to 4 exhibited 1=best, 2=very good or 3=good activity and d) if there were any negative effects.

Of the 15 subjects, 14 subjects were of the subjective opinion that all silicone pads were well tolerated. A subject described the tolerability as good more or less.

Of the 15 subjects, 13 subjects were of the subjective opinion that a visible reduction in wrinkles and/or other positive effects were noted with respect to all silicone pads. Two subjects described that the visible wrinkle reduction and/or other positive effects occurred only with respect to silicone pads 2 to 3, while they were uncertain with respect to silicone pad 1.

With regard to the efficacy of the silicone pads 1 to 4, the subjective observations of the subjects are summarized in Table 2.

TABLE 2
Result of the efficacy study.
Observed efficacy

| | efficacy observed | | |
|---|---|---|---|
| Silikonpad No. | 1 (best) | 2 (very good) | 3 (good) |
| 1 | — | 5 | 10 |
| 2 | 15 | — | — |
| 3 | 4 | 9 | 2 |
| 4 | 1 | 8 | 6 |

All 15 subjects reported that, regarding application of the silicone pads, negative effects were not detectable.

The empirical efficacy study proves that the silicone pads on the skin are well tolerated and exhibit no negative effects. The silicone pads having hyaluron as the active ingredient were demonstrated to have the best efficacy against wrinkling. Moreover, the silica pads supplied with collagen and, to a lesser extent, with aloe vera showed good efficacy. The silicone pads without active ingredient (blanks) exhibited the worst efficacy with respect to wrinkling.

Without wishing to be confined by any theory, the inventors of the present invention hypothesize that, due to the close or complete adherence of the silicone pad to the skin, the subjects slightly sweat such that the hyaluron or other active ingredients is detached due to the limited thickness of the adhesive layer and is eluted from the silicone material by the sweat. In this way, the silicone pad acts as a long-term and durable storage device on the skin surface and enables the achievement of the desired effect over the entire period of wearing, when utilized as a reusable product, even in the course of repeated or temporally interrupted treatment.

Without wishing to be confined by any theory, the inventors of the present invention hypothesize that in terms of efficacy of scar remission, the silicone pads of the present invention act by the following mechanism of action. By applying the silicone pad, a higher skin temperature may be achieved at which the physiological process of scar healing progresses faster. Alternatively, by applying the silicone pad, a higher humidity may be achieved enabling faster remission of the scar tissue by this process. Moreover, the process may be enhanced by the release of appropriate drugs.

### Example 4: Studies on Skin Compatibility and Skin Tightening

In an empirical efficacy study, silicone pads according to the invention were tested in a total of 25 subjects aged 35 to 64 years. Silicone pads shaped as décolleté pad supplied with hyaluron were tested. The total weight of the silicone pad was 28 grams with 11 mg of added hyaluron.

The silicone pads were brought into overnight contact with the skin during a period of 14 consecutive days. The region of contact was the décolleté. Thereafter, the subjects were asked for their subjective opinion as to whether the silicone pads where tolerable and could be used without problems. Skin tightening was tested using a Visioscan VC 98 comparing pre- and post-application values.

No negative effects regarding the application of the silicone pads were encountered by any of the 25 subjects enrolled in the study. In particular, neither dryness nor reddening of the skin, as well as no unpleasant feeling on the skin due to the silicone pads were reported.

This empirical study of efficacy proves that the silicone pads supplied with active ingredients (here: hyaluron) are well tolerated on the skin, produce a measurable and sustainable skin smoothing and exhibited no negative effects.

Subjects stated that the product did not peel off the skin when worn overnight. The pad smoothed the skin on the décolleté and the skin attained a pleasant smoothness overnight.

Skin imperfections and furrows were visibly removed overnight.

Skin smoothness as measured by Visioscan VC 98 showed that it was smoothed, on average, by 19%. Skin smoothing was observed equally in all subjects.

### LIST OF REFERENCE NUMBERS

1. silicone pad
2. adhesive layer
3. silicone body
4. back of the hand
5. metacarpophalangeal joint
6. proximal interphalangeal joint
7. hinge region

1-15. (canceled)

16. A silicone pad for adhering to the skin, wherein the silicone pad comprises at least one active ingredient suitable for cosmetic skin care or for preventing skin aging.

17. The silicone pad according to claim 16, wherein the silicone pad comprises an adhesive layer and a silicone body, wherein the adhesive layer consists essentially of silicone and contains the active ingredient.

18. The silicone pad according to claim 16, wherein the silicone pad is configured to rest on a back of a hand.

19. The silicone pad of claim 16, wherein the silicone pad has a weight sufficient to flatten the skin when the silicone pad is placed on the skin of a subject.

20. The silicone pad according to claim 16, wherein the active ingredient comprises nutrients.

21. The silicone pad according to claim 16, wherein the active ingredient is selected from the group consisting of vitamins, lipids, amino acids, polypeptides, antibiotics or their prodrugs, antioxidants, coenzymes, polysaccharides, and hydroxycarboxylic acids.

22. The silicone pad according to claim 16, wherein the active ingredient is selected from the group consisting of: vitamin A; vitamin C; vitamin E; vitamin B3; vitamin B5; vitamin B9; vitamin B12; coenzyme 10; arbutin; high and low molecular weight hyaluron; high and low molecular weight hyaluronic acid; collagen; b-resorcinol; 4-n-butylresorcinol; glycolic acid; salicylic acid; spirulina; chlorella; caviar extract; dimethylaminoethanol; shea butter; coconut oil; phytohormones; phytoestrogens; isoflavones; lignans; amino acids; oligopeptides; polypeptides; pentapeptides; glycerol; alpha-lipoic acid; and polyphenols.

23. The silicone pad according to claim 16, wherein the active ingredient is present in the silicone pad at a concentration in the range of about 0.5 to 3.0% (w/w), 1.0 to 2.0% (w/w), or about 1.6% (w/w).

24. The silicone pad according to claim 16, wherein the active ingredient is present in the silicone pad at a concentration in the range of 0.1 to 9.9% (w/w), 0.5 to 7.0% (w/w), or about 1.0% (w/w).

25. The silicone pad according to claim 16, wherein the silicone pad is configured for resting on a back of a hand, wherein the silicone pad is suitable for tightening wrinkles on the back of the hand.

26. The silicone pad according to claim 25, wherein the silicone pad comprises hinges.

27. A method for manufacturing a silicone pad for adhering to the skin comprising at least one active ingredient for cosmetic skin care or for preventing skin aging, comprising the step of applying an active ingredient to a silicone composition to produce a silicone pad.

28. The method for producing a silicone pad according to claim 27, further comprising the steps of: a) mixing an active ingredient with a silicone composition to produce an adhesive layer; and b) attaching the adhesive layer to a silicone body to produce the silicone pad.

29. A cosmetic method for smoothing the skin of a subject or for inducing scar remission in a subject, comprising applying a silicone pad according to claim 1 to the skin of the subject.

30. A method of treatment for treating a skin wound of a subject, comprising applying a silicone pad according to claim 1 to the skin of the subject.

31. A method of treatment for treating a nicotine addiction of a subject, comprising applying a silicone pad according to claim 1 to the skin of the subject.

32. The method of treatment according to claim 31, wherein the active ingredient is delivered to the skin on a regular basis.
